(19) United States
(12) Patent Application Publication
(10) Pub. No.: US 2014/0302543 A1
(43) Pub. Date: Oct. 9, 2014

(54) STRATEGY FOR THE ASSAY OF ENZYME AND ENZYME MIXTURES FOR THE HYDROLYSIS LIGNO-CELLULOSIC BIOMASS

(71) Applicant: C5-6 Italy S.R.L, Tortona (IT)

(72) Inventors: Laura Volpati, Tortona (IT); Stefano Paravisi, Tortona (IT); Beatriz Rivas Torres, Arenzano (IT)

(73) Assignee: C5-6 Italy S.R.L.

(21) Appl. No.: 14/359,484

(22) PCT Filed: Nov. 22, 2012
(86) PCT No.: PCT/IB2012/056626

§ 371 Date: May 20, 2014

(30) Foreign Application Priority Data
Nov. 23, 2011 (IT) TO2011A001077

(51) Int. Cl. C12Q 1/34

(52) U.S. Cl. CPC C12Q 1/34
USPC 435/18

(57) ABSTRACT

The invention is to a novel method for assaying most or all of the activities of an enzyme or enzyme solution that could be used to convert ligno-cellulosic material into monomeric sugars. The method characterizes enzymes and enzyme mixtures for the hydrolysis of ligno-cellulosic biomass by measuring the protein concentration of an enzyme or enzyme mixture, adding the enzyme or enzyme mixture to a plurality of test vials creating a liquid fraction in the plurality of test vials wherein each test vial in the plurality of test vials contains a different substrate, incubating each test vial for a period of time sufficient to establish the conversion of the substrate in each test vial of the plurality of test vials at the same pH and temperature, while, if necessary, providing at least enough agitation to keep the substrate in suspension, quenching the reaction, and analyzing the optical absorbance of the liquid fraction of at least one test vial of the plurality of test vials.

### BACKGROUND

The selection and characterization of an enzyme or enzyme mixture towards a species of ligno-cellulosic biomass is time consuming and often more an art than science. As more attention is paid to commercializing processes which hydrolyze ligno-cellulosic biomass, there is an increased need for better and more active enzymes. There exists therefore, the need for a method to assay enzymes and enzyme mixtures for the hydrolysis of ligno-cellulosic biomass. Many methods have been developed so far for assaying enzymes or enzyme mixtures on a laboratory scale. Typically, the methods are based on the assay of the enzyme or enzyme mixture on a set of exemplary substrates, different from the real ligno-cellulosic biomass. Thereby, the assay of the enzyme or enzyme mixture on a limited set of exemplary substrates may conduct to the evaluation of enzyme activities not fully representative of the enzyme or enzyme mixture for the hydrolysis of the ligno-cellulosic biomass. On the contrary, the use of a large set of exemplary substrates may lead to a set of redundant information. In some cases, expensive substrates are suggested and used, increasing the cost of the assay test. For reducing the cost of the assay of enzyme mixture on an industrial scale, it is needed to minimize the amount of assay tests that must be performed for the assay of the enzyme or enzyme mixture being representative of the hydrolysis of the ligno-cellulosic biomass. Moreover, there is the need to perform a large amount of tests in short time and in a routinely or automatic manner. Finally, the assay test should require small amounts of possibly inexpensive substrates and reagents.

There is therefore the need of methods for the assaying of enzymes and enzyme mixtures for the hydrolysis of ligno-cellulosic biomass which can be performed in a time efficient manner and at a low cost separately from the hydrolysis of the ligno-cellulosic biomass

### SUMMARY

This specification discloses a method for assaying enzymes and enzyme mixtures for the hydrolysis of ligno-cellulosic biomass. The method disclosed in this specification includes the steps of; a) measuring the protein concentration of an enzyme or an enzyme mixture, b) adding the enzyme or the enzyme mixture to a plurality of test vials creating a liquid fraction in the plurality of test vials wherein each test vial in the plurality of test vials contains a different substrate, c) incubating simultaneously each test vial for a period of time sufficient to establish the conversion of the substrate in each test vial of the plurality of test vials at the same pH and temperature, while, if necessary, providing at least enough agitation to keep the substrate in suspension, d) quenching the reaction, and e) analyzing the optical absorbance of the liquid fraction of at least one test vial of the plurality of test vials.

This specification further discloses that at least one test vial of the plurality of test vials contains a filter paper substrate.

This specification further discloses that at least one test vial of the plurality of test vials contains a CMC substrate.

This specification further discloses that at least one test vial of the plurality of test vials contains a salicin substrate.

This specification further discloses that at least one test vial of the plurality of test vials contains a Xylan substrate.

This specification further discloses that at least one test vial of the plurality of test vials contains an Avicel substrate.

This specification further discloses that at least one test vial of the plurality of test vials contains at least one PNP-substrate selected from the group consisting of α-L-arabinofuranoside, β-D-galactopyranoside, β-D-glucuronide, α-D-glucopyranoside, β-D-mannopyranoside, α-D-galactopyranoside.

This specification further discloses that the step of quenching the reaction is done by adding a first reagent which stops the action of the enzyme or enzyme mixture on filter paper to the at least one test vial containing a filter paper substrate.

This specification further discloses that the step of quenching the reaction is done by adding a second reagent which stops the action of the enzyme or enzyme mixture on CMC to the at least one test vial containing a CMC substrate.

This specification further discloses that the step of quenching the reaction is done by adding a third reagent which stops the action of the enzyme or enzyme mixture on salicin to the at least one test vial containing a salicin substrate.

This specification further discloses that the step of quenching the reaction is done by adding a fourth reagent which stops the action of the enzyme or enzyme mixture on Avicel to the at least one test vial containing an Avicel substrate.

This specification further discloses that the step of quenching the reaction is done by adding a fifth reagent which stops the action of the enzyme or enzyme mixture on Xylan to the at least one test vial containing a Xylan substrate.

This specification further discloses that the step of quenching the reaction is done by adding a sixth reagent which stops the action of the enzyme or enzyme mixture on at least one of the six different PNP-substrates to the at least one test vial containing one of the six different PNP-substrates.

This specification also discloses that the first reagent, the second reagent, the third reagent, the fourth reagent and the fifth reagent may be the same reagent and that the same reagent may comprise dinitrosalicylic acid.

This specification further discloses that the optical absorbance of the liquid fraction of the at least one test vial may be analyzed at a unique wavelength.

This specification also discloses that the concentration of each one of the substrates in the test vials, expressed in grams of substrate per 100 ml of liquid fraction, may be in a range of 0.5 to 10, preferably of 0.5 to 7, more preferably of 0.5 to 6, and most preferably of 1 to 5.

This specification further discloses that at least one of the substrates may be present in a homogeneous slurry form.

This specification also discloses that the addition of the enzyme step (b) may be preceded by measuring the protein concentration of the enzyme or enzyme mixture.

This specification further discloses that the ratio of the amount of protein of the enzyme or enzyme mixture, expressed in milligrams, and the amount of each of the substrates, expressed in grams, may be in a range of 0.01 to 5, preferably of 0.02 to 4, more preferably of 0.05 to 3, and most preferably of 0.1 to 2.

This specification also discloses that there may be three test vials containing a filter paper substrate, and/or three test vials containing a CMC substrate, and/or three test vials containing a salicin substrate, and/or three test vials containing a Xylan substrate, and/or three test vials containing an Avicell substrate, and/or three test vials containing at least one PNP-substrate selected from the group consisting of α-L-arabinofuranoside, β-D-galactopyranoside, β-D-glucuronide, α-D-glucopyranoside, β-D-mannopyranoside, α-D-galactopyranoside.

### DETAILED DESCRIPTION

Described in the specification is a method for assaying all of the activities that compose an enzyme or enzyme solution that could be used to convert ligno-cellulosic material into monomeric sugars. The method as described is useful for the rapid evaluation of enzymes and enzyme mixtures for the hydrolysis of ligno-cellulosic biomass.

According to one aspect of the invention, it is disclosed a method for evaluating an enzyme or enzyme mixture for the hydrolysis of a ligno-cellulosic biomass by testing the enzyme or enzyme mixture on a limited set of commercially available substrates different from the ligno-cellulosic biomass.

According to another aspect of the present invention, the disclosed method defines the limited set of tests to be performed on each substrate for having a significant evaluation of the activities of the enzyme or enzyme mixture during the hydrolysis of a ligno-cellulosic biomass.

According to another aspect of the invention, it is disclosed a method for reducing the time and the amount of substrates and reagents needed in each test.

According to another aspect of the invention, low cost analysis instruments are needed for evaluating the enzyme or enzyme mixture for the hydrolysis of a ligno-cellulosic biomass.

A person skilled in the art would appreciate that all the aspects of the disclosed invention are important for defining a method for the rapid and inexpensive evaluation of an enzyme or enzyme mixture for the hydrolysis of a ligno-cellulosic biomass.

The process follows the steps of; a) optionally measuring the protein concentration of an enzyme or an enzyme mixture, b) adding the enzyme or the enzyme mixture to a plurality of test vials creating a liquid fraction in the plurality of test vials wherein each test vial of the plurality of test vials contains a different substrate, c) simultaneously incubating each test vial for a period of time sufficient to establish the hydrolysis of the substrate in each test vial of the plurality of test vials, d) quenching the reaction, and e) analyzing the optical absorbance of the liquid fraction of at least one test vial of the plurality of test vials.

The test vials are characterized as any vial which is capable of containing a liquid such as a test tube or a flask.

Preferably, each test vial in the plurality of test vials contains only one substrate. Preparing the substrates contained in the plurality of test vials requires preparation of a buffer. Different enzymes should be assayed in different buffers varying in type, composition and pH. Buffers can be prepared as a 1N stock solution which can be diluted to 0.05 M to prepare a working solution. By way of example, but not limitation, a citrate buffer solution may be prepared as a 1N stock solution by mixing preferably 210 g of citric acid monohydrate with 750 ml of ultrapure water and adding between 50 and 60 g of NaOH, the amount of NaOH determined by the amount necessary to reach a pH of 4.5. The 0.05 M working solution is then prepared by mixing 50 ml of the 1N stock solution with 950 ml of ultrapure water and adding 1M CaCl₂ (1 ml) and/or 0.45M DTT (22 ml), depending upon the enzyme characteristics, and adjusting the pH to the required value.

In a preferred embodiment, the concentration of each one of the substrates in the test vials, expressed in grams of substrate per 100 ml of liquid fraction, is in a range of 0.5 to 10, preferably of 0.5 to 7, more preferably of 0.5 to 6, and most preferably of 1 to 5.

In another preferred embodiment, at least one of the substrates is present in a homogeneous slurry form or homogeneous suspension. Inventors have found that, being the use of a small amount of substrates desirable for the rapid and economic evaluation of a large set of enzymes and enzymes mixtures for the hydrolysis of a ligno-cellulosic biomass, the preparation and handling of such a small amount of substrates may conduct to misleading results. The use of substrates in slurry form in a small volume requires that the slurry of the substrate in each test vial is statistically representative of a larger sample. In particular, it is required that the concentration of the substrate in the slurry used in each vial is substantially the same concentration of that one in a reference substrate suspension volume of 1 liter. In this way, it is preserved the condition that the amount of enzymes or enzyme mixture per gram of substrate in each test vials is not affected by the small volume of substrate slurry in the test vials.

The volume of the slurry of the substrates in the test vials may be very small with respect to methods disclosed in the prior art, being less than 2.0 ml, preferably less than 1.0 ml, more preferably less than 0.5 ml, even more preferably less than 0.3 ml, yet even more preferably less than 0.2 ml, being less than 0.1 ml the most preferred value. Preferably, at least one of the substrates contained in at least one of the plurality of test vials comprises a substrate such as a filter paper substrate which is useful for quantifying the cellulose degradation activity of the enzymes or enzyme solutions. Preferably, the filter paper substrate consists of a 15 mg strip of Whatman No. 1 filter paper (0.55×3.29 cm). In an preferred embodiment, three test vials of the plurality of test vials comprise a substrate such as a filter paper substrate which is useful for quantifying the cellulose degradation activity of the enzymes or enzyme solutions.

Preferably, at least one of the substrates contained in at least one of the plurality of test vials comprises a substrate such as an Avicel substrate which is useful for quantifying the exo-cellulase activity of the enzymes or enzyme solutions. Preferably, the Avicel substrate is prepared using 5 g Avicel (Sigma, 11365) in combination with 100 ml of a 50 mM citrate buffer kept in continuous agitation using a magnetic stirrer.

In a preferred embodiment, three test vials of the plurality of test vials comprise a substrate such as an Avicel substrate which is useful for quantifying the exo-cellulase activity of the enzymes or enzyme solutions. Preferably, at least one of the substrates contained in at least one of the plurality of test vials comprises a substrate such as a carboxymethyl cellulose (CMC) substrate which is useful for quantifying the endo-cellulase activity of the enzymes or enzyme solutions. Preferably, the CMC substrate is prepared using 2 g carboxymethyl cellulose CMC 7L2 (SIGMA, 419273) in combination with a 100 ml 50 mM citrate buffer.

In a preferred embodiment, three test vials of the plurality of test vials comprise a substrate such as a carboxymethyl cellulose (CMC) substrate which is useful for quantifying the endo-cellulase activity of the enzymes or enzyme solutions. Preferably, at least one of the substrates contained in at least one of the plurality of test vials comprises a substrate such as a salicin substrate which is useful for quantifying the beta-glucosidase activity of the enzymes or enzyme solutions. Preferably, the salicin substrate is prepared using 1 g salicin (Sigma S0625) in combination with 100 ml 50 mM citrate buffer.

In a preferred embodiment, three test vials of the plurality of test vials comprise a substrate such as a salicin substrate which is useful for quantifying the beta-glucosidase activity of the enzymes or enzyme solutions.

Preferably, at least one of the substrates contained in at least one of the plurality of test vials comprises a substrate such as a Xylan substrate which is useful for quantifying the xylanase activity of the enzymes or enzyme solutions. Preferably, the Xylan substrate is prepared using 2 g Birchwood Xylan (Sigma X0502) in combination with 70 ml ultrapure water heated to boiling with stirring and then cooled to room temperature while adding 5 ml of 1 N buffer stock solution.

In a preferred embodiment, three test vials of the plurality of test vials comprise a substrate such as a Xylan substrate which is useful for quantifying the xylanase activity of the enzymes or enzyme solutions.

At least one of the substrates contained in at least one of the plurality of test vials may also comprise a substrate such as an α-L-arabinofuranoside substrate which is useful for quantifying the arabinase activity of an enzyme or enzyme solution. Preferably, the α-L-arabinofuranoside substrate is prepared using 2.5 mg of P-nitrophenyl α-L-arabinofuranoside (Sigma N3641) in combination with 1.84 ml of 50 mM citrate buffer.

In a preferred embodiment, three test vials of the plurality of test vials comprise a substrate such as an α-L-arabinofuranoside substrate which is useful for quantifying the arabinase activity of an enzyme or enzyme solution.

At least one of the substrates contained in at least one of the plurality of test vials may also comprise a substrate such as a β-D-galactopyranoside substrate which is useful for quantifying the galactosidase activity of an enzyme or enzyme solution. Preferably, the β-D-galactopyranoside substrate is prepared using 2.8 mg P-nitrophenyl β-D-galactopyranoside (Sigma N1252) in combination with 1.86 ml of 50 mM citrate buffer.

In a preferred embodiment, three test vials of the plurality of test vials comprise a substrate such as a β-D-galactopyranoside substrate which is useful for quantifying the galactosidase activity of an enzyme or enzyme solution. At least one of the substrates contained in at least one of the plurality of test vials may also comprise a substrate such as a β-D-glucuronide substrate which is useful for quantifying the glucoronidase activity of an enzyme or enzyme solution. Preferably, the β-D-glucuronide substrate is prepared using 10.8 mg P-nitrophenyl β-D-glucuronide (Sigma N1627) in combination with 6.85 ml of 50 mM citrate buffer.

In a preferred embodiment, three test vials of the plurality of test vials comprise a substrate which is useful for quantifying the glucoronidase activity of an enzyme or enzyme solution.

At least one of the substrates contained in at least one of the plurality of test vials may also comprise a substrate such as a α-D-glucopyranoside substrate which is useful for quantifying the beta-glucosidase activity of an enzyme or enzyme solution. Preferably, the α-D-glucopyranoside substrate is prepared using 8 mg P-nitrophenyl α-D-glucopyranoside (Sigma N1377) in combination with 5.31 ml of 50 mM citrate buffer.

In a preferred embodiment, three test vials of the plurality of test vials comprise a substrate such as a α-D-glucopyranoside substrate which is useful for quantifying the beta-glucosidase activity of an enzyme or enzyme solution.

At least one of the substrates contained in at least one of the plurality of test vials may also comprise a substrate such as a β-D-mannopyranoside substrate which is useful for quantifying the mannosidase activity of an enzyme or enzyme solution. Preferably, the β-D-mannopyranoside substrate is prepared using 8 mg P-nitrophenyl β-D-mannopyranoside (Sigma N1268) in combination with 5.31 ml of 50 mM citrate buffer.

In a preferred embodiment, three test vials of the plurality of test vials comprise a substrate such as a β-D-mannopyranoside substrate which is useful for quantifying the mannosidase activity of an enzyme or enzyme solution.

At least one of the substrates contained in at least one of the plurality of test vials may also comprise a substrate such as a α-D-galactopyranoside substrate which is useful for quantifying the galactosidase activity of an enzyme or enzyme solution. Preferably, the α-D-galactopyranoside substrate is prepared using 8 mg P-nitrophenyl α-D-galactopyranoside (Sigma N0877) in combination with 5.31 ml of 50 mM citrate buffer.

In a preferred embodiment, three test vials of the plurality of test vials comprise a substrate such as a α-D-galactopyranoside substrate which is useful for quantifying the galactosidase activity of an enzyme or enzyme solution. Once the appropriate substrates have been prepared and added to the plurality of test vials, the enzyme or enzyme solution must be added. In order to directly compare different enzyme solutions, it is preferable that each enzyme solution is diluted to the same final enzyme concentration. In some cases, it could be preferable to make more than one dilution to better estimate activity contents.

Preferably, the ratio of the amount of protein of the enzyme or enzyme mixture, expressed in milligrams, and the amount of each of the substrates, expressed in grams, may be in a range of 0.01 to 5, preferably of 0.02 to 4, more preferably of 0.05 to 3, and most preferably of 0.1 to 2. When three test vials of the plurality of test vials comprise a specific substrate, preferably the enzyme or enzyme mixture is present in a concentration of about 0.1, 0.2 and 2 milligrams of protein per grams of substrate, in the three vials respectively. The following procedures are necessary to analyze for quantifying the activity taking place in a test vial containing a filter paper substrate, a CMC substrate, a salicin substrate, a Xylan substrate and/or an Avicel substrate. Preferably, the temperature of the plurality of test tubes is equilibrated by placing them in a thermomixer at a temperature corresponding to the maximum enzyme reaction rate for five minutes before adding the enzyme or enzyme solution. Test tubes containing Avicel substrate are preferably set to 1000 rpm. Once the enzyme or enzyme solution is added to the test tube it is incubated in the thermomixer for a time of preferably between 10 minutes and 60 minutes. Preferably a reagent is added to the enzyme or enzyme mixture to quench the reaction rate, and the resulting combination is incubated at a temperature of preferably 99° C. for a time of preferably at least 7 minutes in order to sufficiently reduce or stop the reaction of the enzyme or enzyme mixture. Preparation of the reagent will depend upon the type of enzyme or enzyme mixture used. Even if each substrate may have a specific reagent for quenching the reaction, preferably the same reagent is used for quenching the reaction occurring in the vials. Preferably, the reagent should be a 3,5-Dinitrosalicylic acid solution. Preparation of the reagent will depend upon the type of enzyme or enzyme mixture in use. Optical absorbance is read at preferably 540 nm.

The following procedures are necessary to analyze for quantifying the activity taking place in a test vial containing a α-L-arabinofuranoside substrate, a β-D-galactopyranoside substrate, a β-D-glucuronide substrate, a α-D-glucopyranoside substrate, a β-D-mannopyranoside substrate, and/or a α-D-galactopyranoside substrate. Preferably, the temperature of the plurality of test tubes is equilibrated by placing them in a thermomixer at a temperature selected to match enzyme optimum for five minutes before adding the enzyme or enzyme solution. The thermomixer should preferably contain a microtitre plate (MTP) adapter and preferably be set to 500 rpm while covering the plate with a plastic foil. Once the enzyme or enzyme solution is added to the test tube it is covered with the plastic foil and incubated in the thermomixer at preferably 50° C. for a time of preferably between 10 minutes and 60 minutes. Reducing the rate of enzyme reaction requires that an amount of reagent be added to the enzyme or enzyme mixture, and that the resulting combination is incubated at preferably 500 rpm at a temperature of preferably 25° C. for a time of preferably at least 10 minutes while maintaining preferably the plate with a plastic foil in order to stop the action of the enzyme or enzyme mixture. Preferably, the reagent should be a 1M Na₂CO₃ solution. Optical absorbance is read at preferably 405 nm.

Quenching the reaction is characterized by sufficiently reducing the rate of reaction so that the analysis of the composition of the vials can be accurately obtained corresponding to the time the vials are removed from the temperature controlled environment. Typically, the reaction rate will be reduced to a rate in the range of 0 to 20% of the rate of the reaction being evaluated. More preferably, the rate will be in the range of 0 to 10%, with 0 to 5% being even more preferred with 0 to 2% being the most preferred.

One method of preparing a reagent may involve the steps of; a) dissolving an amount of distilled water with an amount of dinitrosalicylic acid and an amount of sodium hydroxide, b) adding an amount of Rochelle salts, c) adding an amount of phenol and, d) adding an amount of sodium metabisulfite (Na₂S₂O₅). Preferably the amount of distilled water is 1416 ml. Preferably the amount of dinitrosalicylic acid is 10.6 g. Preferably the amount of sodium hydroxide is 19.8 g. Rochelle salts may include, but are not limited to sodium potassium tartrate and KNaC₄H₄O₆.4H₂O. Rochelle salts are preferably added in an amount of 306 g. Phenols are preferably added in an amount of 7.6 ml. Sodium metabisulfite (Na₂S₂O₅) is preferably added in an amount of 8.3 g.

Another method of preparing a reagent may involve preparing an Na₂CO₃ solution by dissolving an amount of Na₂CO₃ in ultrapure water to a final volume. Preferably the amount of Na₂CO₃ is 10.6 g. Preferably the final volume is 100 ml.

After quenching the reaction the sample may be cooled in ice for a time of preferably 10 minutes.

If dilution is needed, the step of analyzing the optical absorbance of the liquid fraction of at least one test vial of the plurality requires placing preferably a portion of the sample directly in a well of preferably a 96-well plate and adding ultrapure water and mixing the sample vigorously to obtain a properly diluted sample.

We claim:

1-15. (canceled)

16. A method for characterizing enzyme and enzyme mixtures for the hydrolysis of ligno-cellulosic biomass comprising the steps of
a. Adding the enzyme or the enzyme mixture to a plurality of test vials creating a liquid fraction in the plurality of test vials, wherein there is at least one test vial containing a filter paper substrate, at least one test vial containing a CMC substrate, at least one test vial containing a salicin substrate, at least one test vial containing a Xylan substrate, at least one test vial containing an Avicell substrate, and at least one test vial containing at least one PNP-substrate selected from the group consisting of α-L-arabinofuranoside, β-D-galactopyranoside, β-D-glucuronide, α-D-glucopyranoside, β-D-mannopyranoside, α-D-galactopyranoside,
b. Incubating simultaneously each test vial for a period of time sufficient to establish the hydrolysis of the substrate in each test vial of the plurality of test vials at the same pH and temperature, while, if necessary, providing at least enough agitation to keep the substrate in suspension,
c. Quenching the reaction,
d. Analyzing the optical absorbance of the liquid fraction of at least one test vial of the plurality.

17. The process of claim 16, wherein the step of quenching the reaction is done by adding
a first reagent which stops the reaction of the enzyme or enzyme mixture on filter paper to the at least one test vial containing filter paper,
a second reagent which stops the action of the enzyme or enzyme mixture on CMC to the at least one test vial containing CMC,
a third reagent which stops the action of the enzyme or enzyme mixture on salicin to the at least one test vial containing salicin,
a fourth reagent which stops the action of the enzyme or enzyme mixture on avicell to the at least one test vial containing avicell,
a fifth reagent which stops the action of the enzyme or enzyme mixture on xylan to the at least one test vial containing xylan,
a sixth reagent which stops the action of the enzyme or enzyme mixture on at least one of 6 different PNP-substrates to the at least one test vial containing the at least one of the 6 different PNP-substrates.

18. The process of claim 17, wherein the first reagent, the second reagent, the third reagent, the fourth reagent and the fifth reagent are the same reagent.

19. The process of claim 18, wherein the same reagent comprises dinitrosalicylic acid.

20. The process of claim 18, wherein the optical absorbance of the liquid fraction of the at least one test vial is analyzed at a unique wavelength.

21. The process of claim 16, wherein the concentration of each one of the substrates in the test vials, expressed in grams of substrate per 100 mL of fraction, is in a range selected from the group consisting of 0.5 to 10, 0.5 to 7, 0.5 to 6, and 1 to 5.

22. The process of claim 16, wherein at least one of the substrates is present in a homogeneous slurry form.

23. The process of claim 16, wherein the addition of the enzyme step (b) is preceded by measuring the protein concentration of the enzyme or enzyme mixture.

24. The process of claim 23, wherein the ratio of the amount of protein of the enzyme or enzyme mixture, expressed in milligrams, and the amount of each of the substrates, expressed in grams, is in a range selected from the group consisting of 0.01 to 5, 0.02 to 4, 0.05 to 3, and 0.1 to 2.

25. The process of claim 16, wherein there are three test vials containing a filter paper substrate.

26. The process of claim 16, wherein there are three test vials containing a CMC substrate.

27. The process of claim 16, wherein there are three test vials containing a salicin substrate.

28. The process of claim 16, wherein there are three test vials containing a Xylan substrate.

29. The process of claim 16, wherein there are three test vials containing an Avicell substrate.

30. The process of claim 16, wherein there are three test vials containing at least one PNP-substrate selected from the group consisting of α-L-arabinofuranoside, β-D-galactopyranoside, β-D-glucuronide, α-D-glucopyranoside, β-D-mannopyranoside, α-D-galactopyranoside.
